(19) United States
(12) Patent Application Publication
(10) Pub. No.: US 2009/0198214 A1
(43) Pub. Date: Aug. 6, 2009

(54) Butterfly Needle Devices and Methods Relating Thereto

(76) Inventor: Istvan Bognar, Greenville, SC (US)

Correspondence Address: DORITY & MANNING, P.A., POST OFFICE BOX 1449, GREENVILLE, SC, 29602-1449, US

(21) Appl. No.: 12/364,157

(22) Filed: Feb. 2, 2009

Related U.S. Application Data
(60) Provisional application No. 61/025,419, filed on Feb. 1, 2008.

(51) Int. Cl. A61M 5/32

(52) U.S. Cl. 604/506; 604/272

(57) ABSTRACT

In accordance with one embodiment of the present disclosure, a butterfly needle device is described. The device comprises a needle having a proximal end and a distal tip, a base positioned over the needle, a first wing and a second wing each extending from the base and being configured to move between a generally abducted position and a generally adducted position, a spacer element extending from the first wing, a sleeve positioned over the needle, and a bridge element connecting the sleeve to the base.

### CROSS REFERENCE TO RELATED APPLICATION

The present application is based on and claims priority to U.S. Provisional Application Ser. No. 61/025,419 having a filing date of Feb. 1, 2008, which is incorporated by reference herein.

### BACKGROUND

Accidental needle sticks can be painful and can transmit disease and cause infection. A single case of transmitted disease or infection from an accidental needle stick can result in great cost.

One type of needle device that can cause accidental needle sticks is a butterfly needle. A butterfly needle is a needle that has wings on either side which can aid in manipulating and stabilizing the needle during insertion and removal. Many devices have been created to eliminate or reduce the risk of accidental needle sticks from butterfly needles. However, none of these devices satisfactorily protect against accidental needle sticks prior to insertion, during insertion, and after removal of the needle in an efficient and cost effective manner.

Thus, a need exists for a device that protects against accidental needle sticks from butterfly needles. Methods relating to such a device would also be desirable.

### SUMMARY

In accordance with one embodiment of the present disclosure, a butterfly needle device is described. The device comprises a needle having a proximal end and a distal tip, a base positioned over the needle, a first wing and a second wing each extending from the base and being configured to move between a generally abducted position and a generally adducted position, a spacer element extending from the first wing, a sleeve positioned over the needle, and a bridge element connecting the sleeve to the base. When, the first wing and the second wing are in a generally abducted position, the sleeve covers the distal tip of the needle. When the first wing and the second wing are in a generally adducted position, the spacer element actuates the bridge element causing the bridge element to pull the sleeve toward the proximal end of the needle, resulting in the distal tip of the needle being exposed.

In another embodiment of the present disclosure, a butterfly needle device is described. The device comprises a needle having a proximal end and a distal tip, a base positioned over the needle, a first wing and a second wing each extending from the base and being configured to move between a generally abducted position and a generally adducted position, a spacer element extending from the first wing, the circumference of the spacer element decreasing as the spacer element extends from the second wing, a sleeve positioned over the needle, and a bridge element connecting the sleeve to the base. The second wing defines an opening. When the first wing and the second wing are in a generally abducted position, the sleeve covers the distal tip of the needle. When the first wing and the second wing are in a generally adducted position, the spacer element actuates the bridge element causing the bridge element to pull the sleeve toward the proximal end of the needle, resulting in the distal tip of the needle being exposed and the spacer element extends through the opening of the second wing.

In yet another embodiment of the present disclosure, a method for using a butterfly needle device is described. The method comprises positioning a butterfly needle device for insertion in a patient, the device including a needle having a proximal end and a distal tip, a base positioned over the needle, a first wing and a second wing each extending from the base and being configured to move between a generally abducted position and a generally adducted position, a spacer element extending from the first wing, a sleeve positioned over the needle, and a bridge element connecting the sleeve to the base. The first wing and the second wing are moved from a generally abducted position wherein the sleeve covers the distal tip of the needle to a generally adducted position wherein the spacer element actuates the bridge element causing the bridge element to pull the sleeve toward the proximal end of the needle, resulting in the distal tip of the needle being exposed.

Other features and aspects of the present disclosure are discussed in greater detail below.

### BRIEF DESCRIPTION OF THE DRAWINGS

A full and enabling disclosure, including the best mode thereof, directed to one of ordinary skill in the art, is set forth more particularly in the remainder of the specification, which makes reference to the appended figures in which:

FIG. 1 illustrates a perspective view of a device in an abducted position in accordance with certain embodiments of the present disclosure;

FIG. 2 illustrates a cross-sectional view of a device in an abducted position in accordance with certain embodiments of the present disclosure;

FIG. 3 illustrates a perspective view of a device in an adducted position in accordance with certain embodiments of the present disclosure; and

FIG. 4 illustrates a cross-sectional view of a device in an adducted position in accordance with certain embodiments of the present disclosure.

Repeat use of reference characters in the present specification and drawings is intended to represent same or analogous features or elements of the disclosure.

### DETAILED DESCRIPTION

Reference now will be made in detail to various embodiments of the disclosure, one or more examples of which are set forth below. Each example is provided by way of explanation of the disclosure, not limitation of the disclosure. In fact, it will be apparent to those skilled in the art that various modifications and variations can be made in the present disclosure without departing from the scope or spirit of the disclosure. For instance, features illustrated or described as part of one embodiment, can be used on another embodiment to yield a still further embodiment. Thus, it is intended that the present disclosure covers such modifications and variations as come within the scope of the appended claims and their equivalents.

The present disclosure is generally directed to butterfly needle devices and methods relating thereto. As described above, a butterfly needle can very generally be considered a needle that has wings on either side which can aid in manipulating and stabilizing the needle during insertion and removal. In certain embodiments of the present disclosure, devices and methods are provided that allow a user to avoid accidental needle sticks prior to insertion, during insertion, and after removal of a butterfly needle. Reduction in accidental needle sticks can reduce the transmission of disease and infections and thereby reduce costs associated with the same.

Referring to FIG. 1, a butterfly needle device 100 in accordance with the present disclosure is illustrated. The device 100 includes needle 110, base 112, wings 102, sleeve 104, and tubing 114. Needle 110 can be any suitable needle as would be known in the art. Typically, the butterfly needle device 100 of the present disclosure can be utilized for phleboclysis, hemodialytic treatments, venous catheterizations and the like. However, it should be understood that the butterfly needle device of the present disclosure can be utilized for any procedure in which fluids are introduced into or withdrawn from a patient.

The needle 110 is generally cylindrical in shape and includes a proximal end 120 and a distal tip 122. The diameter of the needle 110 is indicated by the needle gauge and various needle lengths are available for any given gauge as would be known and appreciated in the art. For instance, in certain embodiments, the needle can be from about 1 cm to about 10 cm in length. Again, however, it should be understood that any suitable needle having any suitable gauge or any suitable length is contemplated by the present disclosure. The distal tip 122 can be bevelled to create a sharp pointed tip so as to allow the needle 110 to easily penetrate the skin of a patient.

Base 112 is illustrated as being situated over needle 110. Base 112 is shown surrounding needle 110 generally adjacent to the proximal end 120 of needle 110. However, base 112 can be any suitable size or shape and is not necessarily required to completely surround needle 110. For instance, base 112 can cover only a portion of needle 110, such as that portion from which it is intended for wings to extend. In certain embodiments, base 112 can be from about 0.5 cm to about 5 cm in length. Base 112 can be affixed to needle 110 by any method as would be known in the art such as adhesives or the like or can be formed directly on needle 110 and allowed to cure and harden. Base 112 can be formed from any suitable material, such as plastic, elastomeric materials, such as flexible polymers, metals, or combinations thereof. In addition, base 112 can be biocompatible.

As illustrated in FIG. 1, wings 102 extend from base 112. Wings 102 include first wing 116 and second wing 118. Wings 102 can be any suitable size or shape and the outlines of first wing 116 and second wing 118 can be generally symmetrical, as illustrated, or can be non-symmetrical. For instance, in certain embodiments, each wing 116, 118 can be from about 0.5 cm to about 5 cm in length.

First wing 116 includes spacer element 108 which extends from first wing 116. Spacer element 108 can be attached to first wing 116 by any suitable method as would be known in the art such as adhesive or the like or can be integrally formed with first wing 116. Spacer element 108 is illustrated as generally wedge shaped and the circumference of spacer element decreases as spacer element extends from first wing 116. However, as will be described in more detail, spacer element can be any suitable size and shape in accordance with the present disclosure.

Second wing 118 can define an opening 126, as illustrated. If present, the opening 126 can be of suitable size and shape to accommodate a portion of spacer element 108.

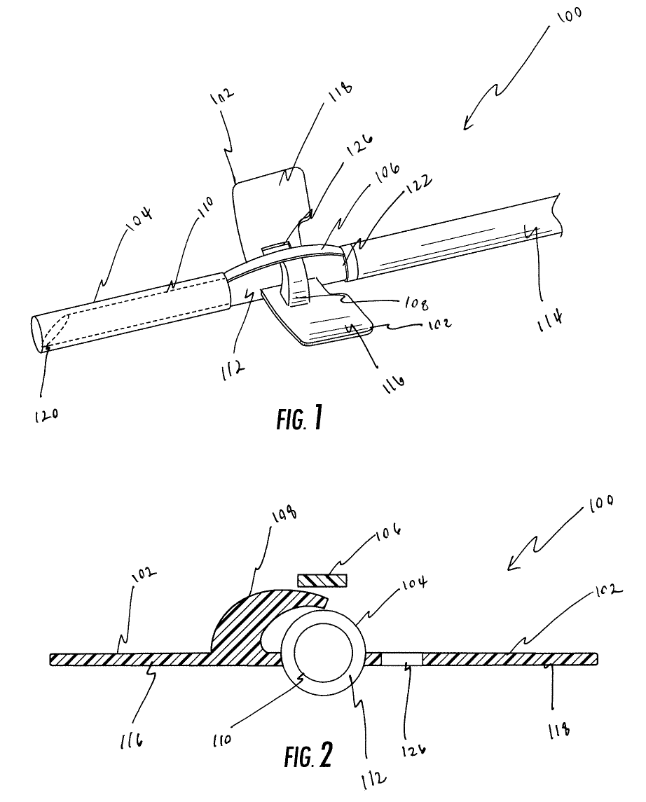

In this regard, wings 102 can be designed to move between a generally abducted position, in which the wings spread apart and a generally adducted position, in which the wings are brought closer together. Referring to FIG. 1, wings 102 are in a generally abducted position with each wing generally flat. In such an arrangement, first wing 116 and second wing 118 can be displaced from one another by about 180 degrees, such as illustrated in FIGS. 1 and 2. In certain embodiments, wings 102 can be configured to have a tendency to stay in an abducted position, such as by molding the wings into such a position.

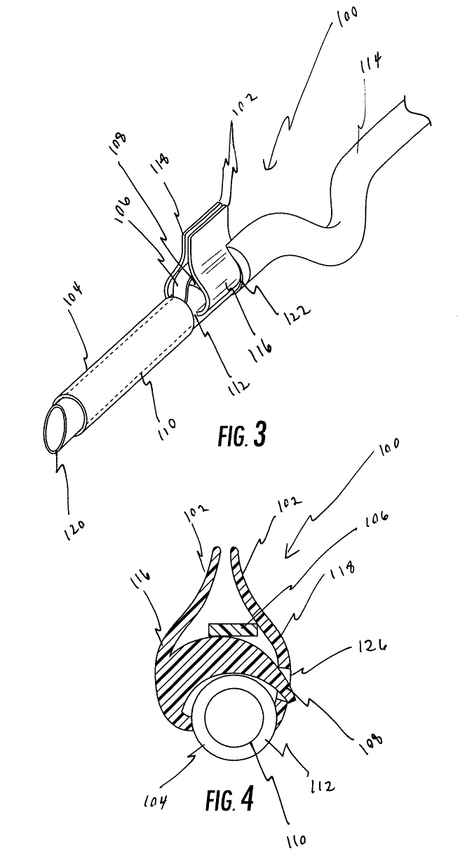

Turning to FIG. 3, wings 102 are in a generally adducted position with first wing 116 and second wing 118 bending towards one another. An adducted position can include any position in which the wings 102 are brought closer together, even if the wings do not come into contact, such as shown in FIGS. 3 and 4.

Wings 102 can each be affixed to base 112 by any method as would be known in the art such as adhesives or the like or can be formed integrally with base 112. In the alternative, wings 102 can be affixed directly on needle 110, in which case the portions of wings 102 that contact needle 110 would serve as base 112. Wings 102 can each be formed from any suitable material, such as plastic, elastomeric materials, such as flexible polymers, metals, or combinations thereof. In addition, wings 102 can be biocompatible.

Referring again to FIG. 1, sleeve 104 is illustrated as being situated over needle 110. Sleeve 104 is shown surrounding needle 110 generally adjacent to the distal tip 122 of needle 110. However, sleeve 104 can be any suitable size or shape so long as sleeve 104 is capable of covering distal tip 122 of needle 110. Sleeve 104 can be formed from any suitable material, such as plastic, elastomeric materials, such as flexible polymers, metals, or combinations thereof. In addition, sleeve 104 can be biocompatible.

Sleeve 104 is attached to bridge element 106. Bridge element 106 can be formed from any suitable material, such as plastic, elastomeric materials, metals, or combinations thereof. In addition, bridge element 106 can be biocompatible. In certain embodiments, bridge element 106 is configured to have minimal stretch. In this regard, bridge element 106 is configured to pull sleeve 104 toward the proximal end 120 of needle 110 when bridge element 106 is actuated by spacer element 108. As described previously, wings 102 can be designed to move between a generally abducted position, in which the wings spread apart and a generally adducted position, in which the wings are brought closer together. When the wings 102 are moved to a generally adducted position, spacer element 108 can actuate bridge element 106 causing bridge element 106 to pull sleeve 104 toward proximal end 120 of needle 110.

Sleeve 104 is designed to move between a position in which sleeve 104 covers the distal tip of needle 110 and a position in which the distal tip of needle 110 is exposed. This can be accomplished any number of ways, all of which are contemplated by the present disclosure. For example, sleeve 104 can slide along needle 110 to expose distal tip of needle 110. In certain embodiments, a space can exist between sleeve 104 and body 112 and/or wings 102 to permit sleeve to move. In certain embodiments, sleeve 104 can compress when pulled by bridge element 106. In certain embodiments, the end portion of sleeve 104 nearest to the distal tip of needle 110 can roll over itself when pulled by bridge element 106. In addition, sleeve 104 can include a seal that is configured to break the first time the bridge element 106 pulls the sleeve toward the proximal end of the needle, resulting in the distal tip of the needle being exposed. In this manner, the needle can remain sterile until its first use.

Turning to FIG. 2, certain embodiments of the mechanism by which bridge element 106 pulls sleeve 104 are illustrated. Spacer element 108 has a generally wedge shape, a portion of which is curved underneath bridge element 106 when wings 102 are in a generally abducted position. In certain embodiments, spacer element 108 can contact bridge element 106 when wings 102 are in a generally abducted position. The circumference of spacer element 108 decreases as spacer element 108 extends from first wing 116 so that the portion of spacer element 108 underneath bridge element 106 has the smallest circumference. When wings 102 are moved into an adducted position, spacer element 108 actuates bridge element 106 by rotating beneath bridge element 106 and pushing bridge element 106 outward, causing sleeve 104 to be pulled. In this manner, sleeve 104 can be configured to cover the distal tip 122 of the needle 110 when the wings 102 are in an abducted position and allow the distal tip 122 of the needle 110 to be exposed when the wings are in an adducted position.

Referring again to FIG. 1, in addition to being attached to sleeve 104, bridge element 106 is attached to base 112. Bridge element can be attached to base 112 and sleeve 104 by any method as would be known in the art such as adhesives or the like or can be formed integrally with one or more of base 112, sleeve 104, and one or both wings 102. In certain embodiments, bridge element 106 can be affixed directly on needle 110, in which case the portion of bridge element 106 that contacts needle 110 would serve as base 112. By being attached to something other than sleeve 104, bridge element 106 has an anchor from which to pull sleeve 104.

Tubing 114 can be attached to base 112. Tubing 114 can be attached to base 112 by any method as would be known in the art such as adhesives or the like or can be formed integrally with one or more of base 112, sleeve 104, bridge 106, and one or both wings 102. In certain embodiments, tubing 114 can be attached directly to needle 110, in which case the portion of tubing 114 that contacts needle 110 would serve as base 112. Tubing 114 can be formed from any suitable material, such as plastic, elastomeric materials, metals, or combinations thereof. In addition, tubing 114 can be biocompatible. Tubing 114 can be connected to a universal Luer-lock, which in turn can be connected to an IV set, blood collection holder, syringe, or the like.

It should be understood that the illustrated device is but only one of numerous examples of devices contemplated by the present disclosure. For example, spacer element could be designed to actuate bridge element by pushing or pulling bridge element causing bridge element to twist and pull on sleeve. In such embodiments, spacer element can have a completely different shape from that illustrated. Similarly, spacer elements can be present on both wings in certain embodiments with both wings optionally defining openings.

Turning to FIGS. 1-4, a method for using a butterfly needle device is illustrated in accordance with the present disclosure. The method can be completed by any trained user of the device, such as a nurse or other medical staff, or even self-administered by a patient. The method includes positioning the device 100 in a position for insertion. The wings 102 of the device can be grasped and moved from the generally abducted position shown in FIGS. 1 and 2 to the generally adducted position shown in FIGS. 3 and 4. In this manner, the sleeve 104 that covers the distal tip 122 of the needle 110 is pulled back exposing the distal tip 122 only when the wings are moved to the adducted position. The needle can then be inserted into the patient.

Once the needle has been inserted, the wings can continue to be held in position or can optionally be permitted to return to a generally abducted position. Because the needle is inserted however, the sleeve will be blocked by the patient's skin from covering the needle. The generally abducted configuration allows the wings to be taped to the patient to stabilize the device while fluids are introduced into or withdrawn from the patient. Alternatively, the wings can be folded flat while still in an adducted position and taped as well. When the fluid transfer is complete, tape can be removed, if necessary, and the needle can be removed from the patient by grasping the wings of the device and moving them from the generally adducted position to the generally abducted position, if they are not already in such a position. Once the needle is removed, the sleeve will return to cover the exposed needle tip. Accidental needle sticks can be greatly prevented by such a mechanism. In addition, the devices and methods of the present disclosure can protect against accidental needle sticks prior to insertion, during insertion, and after removal of the needle in an efficient and cost effective manner.

In the interests of brevity and conciseness, any ranges of values set forth in this specification are to be construed as written description support for claims reciting any sub-ranges having endpoints which are whole number values within the specified range in question. By way of a hypothetical illustrative example, a disclosure in this specification of a range of 1-5 shall be considered to support claims to any of the following sub-ranges: 1-4; 1-3; 1-2; 2-5; 2-4; 2-3; 3-5; 3-4; and 4-5.

These and other modifications and variations to the present disclosure can be practiced by those of ordinary skill in the art, without departing from the spirit and scope of the present disclosure, which is more particularly set forth in the appended claims. In addition, it should be understood that aspects of the various embodiments can be interchanged both in whole or in part. Furthermore, those of ordinary skill in the art will appreciate that the foregoing description is by way of example only, and is not intended to limit the disclosure.

What is claimed is:

1. A butterfly needle device comprising:
a needle having a proximal end and a distal tip;
a base positioned over the needle;
a first wing and a second wing each extending from the base and being configured to move between a generally abducted position and a generally adducted position;
a spacer element extending from the first wing;
a sleeve positioned over the needle; and
a bridge element connecting the sleeve to the base;
wherein when the first wing and the second wing are in a generally abducted position, the sleeve covers the distal tip of the needle, and wherein when the first wing and the second wing are in a generally adducted position, the spacer element actuates the bridge element causing the bridge element to pull the sleeve toward the proximal end of the needle, resulting in the distal tip of the needle being exposed.

2. The device of claim 1, further comprising a tube, the tube being connected to the base.

3. The device of claim 1, wherein the sleeve is generally cylindrical in shape.

4. The device of claim 1, wherein the second wing defines an opening such that the spacer element extends through the opening when the first wing and the second wing are in a generally adducted position.

5. The device of claim 1, wherein the base, the first wing, the second wing, the spacer element, the sleeve, the bridge element, or combinations thereof are formed from a flexible polymer.

6. The device of claim 1, wherein the circumference of the spacer element decreases as the spacer element extends from the second wing.

7. The device of claim 1, wherein the base, the first wing, and the second wing are integrally formed.

8. The device of claim 1, wherein the first wing and the second wing are configured to come into contact with each other when they are in a generally adducted position.

9. The device of claim 1, wherein the first wing and the second wing have generally the same shape.

10. The device of claim 1, wherein the sleeve comprises a seal configured to break the first time the bridge element pulls the sleeve toward the proximal end of the needle, resulting in the distal tip of the needle being exposed.

11. A butterfly needle device comprising:
a needle having a proximal end and a distal tip;
a base positioned over the needle;
a first wing and a second wing each extending from the base and being configured to move between a generally abducted position and a generally adducted position, the second wing defining an opening;
a spacer element extending from the first wing, the circumference of the spacer element decreasing as the spacer element extends from the second wing;
a sleeve positioned over the needle; and
a bridge element connecting the sleeve to the base;
wherein when the first wing and the second wing are in a generally abducted position, the sleeve covers the distal tip of the needle, and wherein when the first wing and the second wing are in a generally adducted position, the spacer element actuates the bridge element causing the bridge element to pull the sleeve toward the proximal end of the needle, resulting in the distal tip of the needle being exposed, the spacer element extending through the opening of the second wing.

12. The device of claim 11, further comprising a tube, the tube being connected to the base.

13. The device of claim 11, wherein the sleeve is generally cylindrical in shape.

14. The device of claim 11, wherein the base, the first wing, the second wing, the spacer element, the sleeve, the bridge element, or combinations thereof are formed from a flexible polymer.

15. The device of claim 11, wherein the base, the first wing, and the second wing are integrally formed.

16. The device of claim 11, wherein the first wing and the second wing are configured to come into contact with each other when they are in a generally adducted position.

17. The device of claim 16, wherein the first wing and the second wing have generally the same shape.

18. The device of claim 11, wherein the sleeve comprises a seal configured to break the first time the bridge element pulls the sleeve toward the proximal end of the needle, resulting in the distal tip of the needle being exposed.

19. A method for using a butterfly needle device, the method comprising:
positioning a butterfly needle device for insertion in a patient, the device comprising a needle having a proximal end and a distal tip, a base positioned over the needle, a first wing and a second wing each extending from the base and being configured to move between a generally abducted position and a generally adducted position, a spacer element extending from the first wing, a sleeve positioned over the needle, and a bridge element connecting the sleeve to the base;
moving the first wing and the second wing from a generally abducted position wherein the sleeve covers the distal tip of the needle to a generally adducted position wherein the spacer element actuates the bridge element causing the bridge element to pull the sleeve toward the proximal end of the needle, resulting in the distal tip of the needle being exposed.

20. The method of claim 19, further comprising:
inserting the needle in the patient; and
removing the needle from the patient whereby the first wing and the second wing are allowed to move from a generally adducted position to a generally abducted position wherein the sleeve covers the distal tip of the needle.
